(19) United States
(12) Patent Application Publication
(10) Pub. No.: US 2009/0035795 A1
(43) Pub. Date: Feb. 5, 2009

(54) METHOD AND COMPOSITION FOR FORMING A UNIFORM LAYER ON A SUBSTRATE

(76) Inventors: Christie DUDENHOEFER, Corvallis, OR (US); Michael J. DAY, Philomath, OR (US); Kenneth WARD, Corvallis, OR (US); Mary E. AUSTIN, Corvallis, OR (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/831,408

(22) Filed: Jul. 31, 2007

(51) Int. Cl. C12Q 1/54; A61B 5/05; B05D 1/00; G01N 33/66; G01N 1/31; B05D 3/00

(52) U.S. Cl. 435/14; 422/68.1; 427/2.13; 427/569; 600/365

(57) ABSTRACT

A method of reducing thickness non-uniformity in a microjet-deposited solids layer is disclosed which comprises depositing onto a substrate by microjet deposition a composition containing a liquid vehicle, at least one reagent; and a polyol which is present at a concentration that enhances thickness uniformity of a solids layer, containing the reagent(s), which is formed when the microjet-deposited composition is dried.

### BACKGROUND

Drop-on-demand (DOD) ink-jet printing is an attractive technique for the controlled repetitive deposition of small quantities of materials onto a substrate. Typical advantages of DOD over other deposition methods include digital control, fine features, edge acuity, multiple inks in close proximity, volume control and placement control. In recent years, the use of ink-jet technology has been extended from printing to a wide variety of additional technical fields, including the fabrication of semiconductors, ceramics, sensors, biopolymer arrays, and for depositing DNA, protein and reagents for biological testing, among other fields of use. Ink-jet dispensing can be an effective means of applying biological or other ingredients to devices, dosage forms or biological test strips. Ink-jet systems are sometimes referred to as “microjet” systems, and the various types of liquid compositions that can be dispensed by microjet, or “microjetted,” are often referred to as “inks.”

The composition and physical properties of an ink-jet ink, particularly viscosity and surface tension, are important factors in ink-jet dispensing technology. The surface tension of an ink composition should be high enough to prevent dripping of the ink from the nozzle. At the same time, the viscosity of the ink should be low enough that it can be ejected by a conventional thermal or bubble ink-jet printhead. Ink-jet inks are, therefore, typically dilute solutions. For instance, thermal ink-jet drop-on-demand devices customarily jet only relatively dilute inks (i.e., total solids loading less than approximately 10% by weight of the ink). Inks for piezoelectric ink-jet devices are usually limited to 20% or less (by weight) solids content.

It may be necessary to jet a dilute ink repeatedly onto a single site on a substrate in order to build up at that deposition site a desired quantity of a dissolved or suspended component of the ink. Moreover, it is desirable in many cases to obtain a uniform layer of solids from multiple droplets deposited at a single deposition site. Working against this goal is the hydrodynamic process in which a drying droplet of a dilute ink tends to deposit its solute at the perimeter of the droplet during drying. This ring forming tendency is commonly known as the “coffee stain” or “coffee ring” effect. It occurs due to the combined action of an increased evaporation rate at the droplet edge, and contact line pinning due to surface irregularities and solute deposition (“self-pinning”). A capillary-driven flow from the droplet center toward the edge compensates for evaporation losses and transports most of the solute toward the contact line. As a result, the solids layer obtained from superimposed application of a series of microjet droplets tends to be in the form of a circle that is thin in the center and much thicker at the perimeter. For the many industrial and scientific processes that utilize ink-jet printing techniques and which require well-defined, uniformly thick ink-jet ink deposits, the elimination of ring formation is of great practical interest.

### SUMMARY

In accordance with certain embodiments of the invention, a method of reducing thickness non-uniformity in a microjet-deposited solids layer is provided which comprises depositing onto a substrate by microjet deposition a composition containing a liquid vehicle, at least one reagent, and a polyol dissolved in the vehicle. The polyol is present at a concentration that enhances thickness uniformity of a solids layer containing the reagent(s) which is formed when the microjet-deposited composition is dried on the substrate.

### BRIEF DESCRIPTION OF THE DRAWING

FIG. 1 is a photomicrograph of pipetted ink drops. Left image ink has no polyol, right image ink contains 2.75% sorbitol, in accordance with an embodiment of the invention.

FIG. 2 is a photomicrograph of pipetted ink drops. Left image ink has no polyol, right image ink contains 2.0% sorbitol, in accordance with an embodiment of the invention.

FIG. 3 is a magnified photograph of reagents, before rapid drying, containing 0.5% sorbitol microjet-deposited on a non-porous support, in accordance with an embodiment of the invention. The support consists of a polymeric substrate (white) with a screen-printed carbon region (black). The yellow-colored reagents cover the entire region in the photograph. The microjet-deposited reagents covering the white polymeric substrate are indicated by arrows.

FIG. 4 is a magnified photograph of the microjet-deposited reagents of FIG. 3, after rapid drying. The microjet-deposited reagents covering the white polymeric substrate are indicated by arrows.

FIG. 5 is a magnified photograph taken before rapid drying of a comparative microjet-deposited reagent containing no sorbitol. The support consists of a polymeric substrate (white) with a screen-printed carbon region (black). The yellow-colored reagents cover the entire region in the photograph. The microjet-deposited reagents covering the white polymeric substrate are indicated by arrows.

FIG. 6 is a magnified photograph taken after rapid drying of the comparative microjet-deposited reagent of FIG. 5, containing no sorbitol. The microjet-deposited reagents covering the white polymeric substrate are indicated by arrows.

FIG. 7 is a magnified photograph of taken before rapid drying of another comparative microjet-deposited reagent containing no sorbitol. The support consists of a polymeric substrate (white) with a screen-printed carbon region (black). The yellow-colored reagents cover the entire region in the photograph. The microjet-deposited reagents covering the white polymeric substrate are indicated by small arrows.

FIG. 8 is a magnified photograph like FIG. 7, except the samples were photographed after rapid drying. Cracks that formed in the deposited reagents upon rapid drying are indicated by large arrows.

FIG. 9 is a graph showing the thickness profile of a representative pipetted reagent containing 2% (by weight) sorbitol and showing the profile of a similar composition without a polyol.

### NOTATION AND NOMENCLATURE

In the following discussion and in the claims:

The terms “including” and “comprising” are used in an open-ended fashion, and thus should be interpreted to mean “comprising, but not limited to . . . ”

The singular forms “a,” “an,” and “the” include plural referents unless the context clearly dictates otherwise. Thus, for example, reference to “a dye” includes reference to one or more of such materials.

“Polyol” refers to an alcohol containing more than two hydroxyl groups, and is sometimes called a polyhydric alcohol or sugar alcohol. Some polyols contain 3-12 carbon atoms substituted with 3-9 hydroxyl groups, such as sorbitol, xylitol, mannitol, maltitol, xylose, glycerol, saccharose and trehalose, for example.

The term “liquid vehicle” is defined to include liquid compositions that can be used to carry active species such as colorants, including pigments and dyes, or biological reagents, such as proteins, enzymes, antibodies, active pharmaceutical ingredients, and small molecules, to a substrate. Liquid vehicles are well known in the art, and a wide variety of liquid vehicle components may be used in accordance with embodiments of the present exemplary system and method. Such liquid vehicles may include a mixture of a variety of different agents, including without limitation, surfactants, solvents, co-solvents, buffers, biocides, viscosity modifiers, sequestering agents, stabilizing agents, and water. Though not liquid per se, the liquid vehicle can also carry other solids, such as polymers, plasticizers, cosolvents and salts.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

### DETAILED DESCRIPTION

A group of polyol-containing ink formulations are disclosed for reducing or preventing the common problem of thickness non-uniformity (“coffee stain effect”) that occurs, upon drying, in dilute inks that have been microjetted onto a substrate. It was found that addition of an amount of a polyol in the range of about 0.1 to about 5.0% by weight to an aqueous-based ink containing other dissolved or suspended solids helps to reduce thickness non-uniformity that occurs upon drying of the ink, compared to the same ink with no polyol additive. Preferably the amount of polyol in the ink is in the range of about 0.1 to about 3% by weight. Applicable polyols include, but are not limited to, those containing 3-12 carbon atoms substituted with 3-9 hydroxyl groups. Some examples are sorbitol, xylitol and mannitol, maltitol, xylose, glycerol, saccharose and trehalose. Some embodiments of the polyol-containing ink formulations provide uniform solids layers with enhanced toughness, flexibility, crack-resistance and resistance to peeling from a substrate, especially when the solids layer formation includes rapid drying of the microjetted ink.

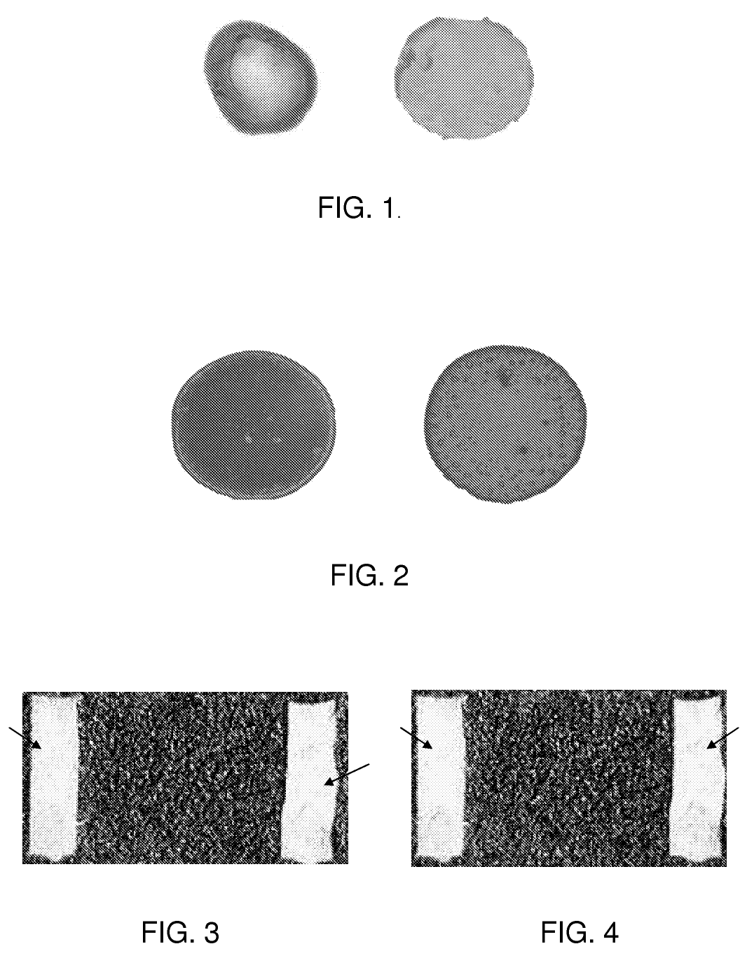

FIG. 1 is a photomicrograph of pipetted ink drops. The image on the left is an ink containing no polyol, while the image on the right is the same ink containing 2.75% (by weight) sorbitol. A similar set of pipetted ink drops were prepared using inks containing no polyol (left image) and 2.0% (by weight) sorbitol (right image), as shown in FIG. 2. The coffee-stain effect is apparent in the inks without sorbitol, which produced a noticeable ring of solids at the margins of the drops after drying.

FIG. 3 is a magnified photograph of reagents, before rapid drying, containing 0.5% sorbitol microjet-deposited on a non-porous support, in accordance with an embodiment of the invention. The support consists of a polymeric substrate (white) with a screen-printed carbon region (black). The yellow-colored reagents cover the entire region in the photograph. The microjet-deposited reagents covering the white polymeric substrate are indicated by arrows. The same microjet-deposited reagent, after rapid drying, is shown in FIG. 4. Notably, the surface of the deposited ink appears unchanged after drying.

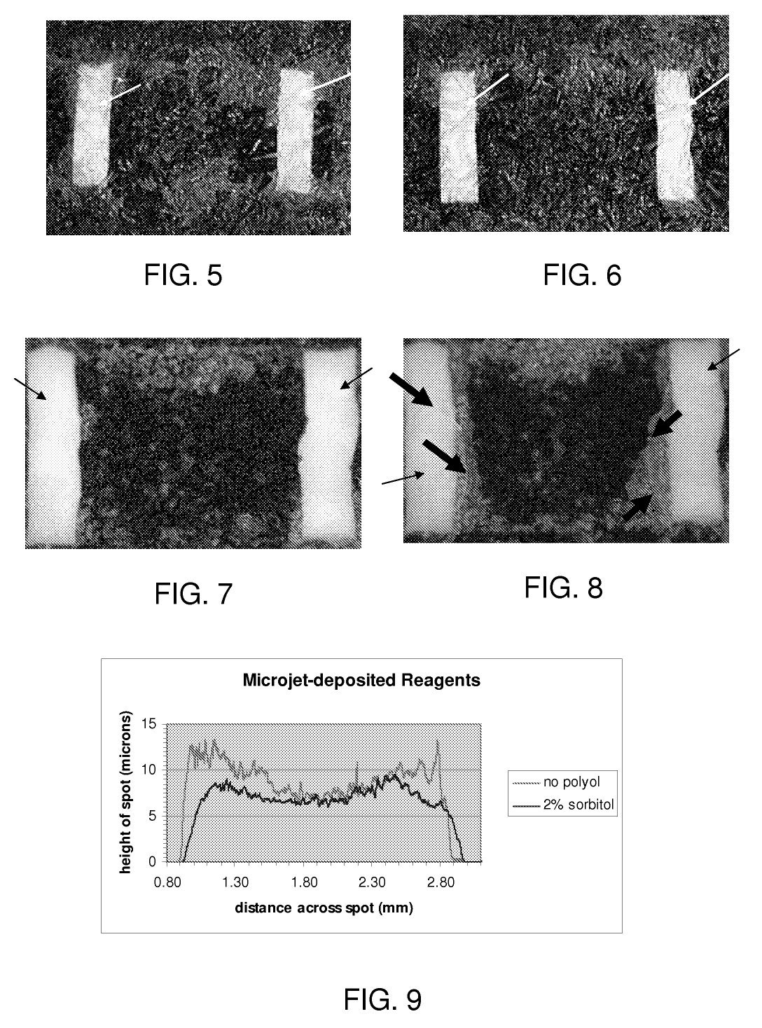

A comparative microjet-deposited reagent containing no sorbitol, before drying, is shown in FIG. 5, in duplicate. The same reagent is shown in FIG. 6, which is a magnified photograph taken after rapid drying. The yellow-colored deposited reagents cover the entire region in the photograph, and are indicated by arrows. Notably, the surface of the deposited reagents show increased roughness after rapid drying of the deposited ink.

FIG. 7 is a magnified photograph of another comparative microjet-deposited reagent containing no sorbitol, before rapid drying of the deposited ink. The same reagent, after rapid drying, is shown in FIG. 8. The yellow-colored deposited reagents cover the entire region in the photograph, and are indicated by small arrows. Cracks that formed in the deposited reagent upon rapid drying are indicated by large arrows.

The thickness profile of a representative pipetted reagent, containing 2% (by weight) sorbitol, after drying, is shown in FIG. 9. The overall thickness of the deposited layer is greater than 0.5 microns. For comparison, the thickness of the same microjet-deposited reagent without a polyol is also shown. The diameters of the deposited samples are about 3 millimeters. The thicker outer margins of the deposited layer is apparent in the sample that lacks the polyol, whereas the thickness of the deposited reagent containing the polyol is more uniform and lacks the thick outer ring. Thickness uniformity of a deposited layer was measured using Scanning White Light Interferometry with a Veeco NT8000. This commercially-available technique measures the height of a surface by comparing the path length of light striking the sample with the path length of a reference beam, at multiple reference beam pathlengths acquired uniformly in time. The light intensities as a function of time comprise an interferogram, which can be utilized to find the thickness between two surfaces. The sample thickness is simultaneously measured throughout the printed region to form an image of the sample thickness, which is then used to determine the thickness uniformity over the printed area.

Embodiments of the polyol-containing ink formulations are particularly suited for printing at high ink volume per area on non-porous substrates to homogenize the thickness of the resulting solids layer, especially to create solid layers greater than 0.5 μm thickness. The addition of a suitable polyol to an ink-jet ink permits a higher-throughput writing system by avoiding the necessity of a large number of passes with fewer drops per pass, in order to create a more uniform layer. Many of these inks will find use in applications where a uniform, thick solids layer is required, and in many industrial and scientific processes in which controlling the distribution of solute during drying is desirable or necessary. For instance, the present methods of forming uniform solids layers are applicable to preparing reagent-containing uniform solids layers on diagnostic test strips.

A representative glucose test strip used for measuring the concentration of glucose in a biological sample comprises a reaction zone comprising ink-jet deposited chemical reagents, one or more working electrodes, one or more counter electrodes, and a dielectric or insulator. Electrical traces lead from the reaction zone to meter contacts on the strip. Electronic circuitry also couples the electrodes to a glucometer. One chemical reagent that is printed on the strip is an electron mediator (e.g., potassium ferricyanide). Another reagent that is applied by ink-jetting comprises one or more enzyme (e.g., glucose oxidase and glucose dehydrogenase). To measure glucose concentration in blood, a blood sample is introduced into the test strip, and flows into the reaction zone which comprises a working electrode, a counter electrode and the microjetted chemical reagents. The blood sample is allowed to react with the reagents in the reaction zone. During the reaction time, glucose is oxidized to form gluconolactone, a step that is catalyzed by the glucose oxidase or glucose dehydrogenase enzyme. The electron removed from the glucose is transferred to the electron mediator, such as potassium ferricyanide. At the end of the test time, a glucometer applies a potential to the working electrode, which draws electrons from the oxidation of ferrocyanide and creates a measurable current. The meter measures the strength of electric current and calculates the glucose concentration. The measured current is proportional to the amount of glucose in the blood.

A microjettable polyol-containing ink for depositing with precision onto glucose test strips a 0.5-10.0 micrometer thick solids layer of reagents for measuring the concentration of glucose in a biological sample comprises at least one enzyme, one or more electron mediators, appropriate buffers, one or more polymers, and at least one surfactant. An example of suitable concentration ranges of these ingredients are given in Table 1.

TABLE 1

| | | Concentration Range |
|---|---|---|
| Ingredient | Example Ingredient | (weight percent) |
| Enzyme | Glucose oxidase | 0.1-3% |
| Electron mediator | Potassium Ferricyanide | 0.1-3% |
| Buffer | Sodium Citrate | 0.1-3% |
| Polymer | Carboxymethyl cellulose | 0.05-3% |
| Surfactant | Triton X-100 | 0.01-1% |
| Polyol | Sorbitol | 0.1-3% |

The reagents are applied to a selected porous or non-porous substrate by ink-jet printing using any suitable ink-jet printer, for example, the ink-jet printing system described in U.S. patent application Ser. No. 11/738,923. To facilitate manufacture, the substrate may be treated with the reagents and then subdivided into smaller portions (e.g., small narrow strips each containing a uniform reagent-containing region) to provide a plurality of identical test strips.

In one alternative embodiment, two or more different microjet-deposited reagents regions may be applied, spaced apart on a test strip, such that the separation between the uniformly deposited reagents and the flow rate characteristics of the porous substrate may be selected to allow adequate reaction times with the liquid sample, during which specific binding can occur, to allow the reagent in the first region to dissolve or disperse in the liquid sample and migrate through the substrate. The two or more reagent regions may be involved in testing for a single component in the liquid sample (analyte), such as glucose, or for multiple components in the liquid sample, such as cholesterol and triglycerides. One or more of the different reagent types may be used to determine a control or calibration number for the reaction of interest. The polyol components may provide additional control over sample or reagent migration on a porous substrate due to the viscosity modifying properties of the polyols.

In another alternative embodiment, multiple reagent types are microjet-deposited onto non-porous substrates, to test for a single component in the liquid sample, such as blood clotting time (INR), or for multiple components in the sample. One or more of the different reagent types may be used to determine a control or calibration number for the reaction of interest. With a non-porous substrate, flow of the liquid sample to each of the reagent types is controlled by features, such as capillary paths. There may be a single capillary path or multiple capillary paths depending on the number of different components or control levels to be tested.

Ink-jet deposition of the reagents using a polyol-containing ink makes possible enhanced sensor performance, including accuracy and precision due to the enhanced uniformity of the dried reagent layer. Other potential advantages of ink-jet reagent deposition include product miniaturization to allow for making a disposable all-in-one meter; continuous flow manufacturing of diagnostic test strips; and greater manufacturing efficiency, with reduced reagent waste and improved serviceability of the test meter.

Ink-jet printing with polyol-containing inks offer potential advantages over other deposition technologies such as screen printing and micropipetting. These include better volumetric precision (<1% CV) and accuracy. In addition, the patterning capability and non-contact printing of ink-jet printing with polyol-containing inks makes possible alignment of deposited reagents to substrate geometries, the use of multiple reagents in close and controlled proximity to one another, and layering of reagents and other chemistries. Still other potential advantages include: “plug-and-play” simplicity by virtue of disposable supplies which reduce cleaning and validation operations. Another potential field of application is the rapid analysis of constituents of blood, or an analysis fluid that contains particulate matter such as cell cultures, particle suspensions, and environmental and industrial samples deposited as uniform thin solids layers. Embodiments of the uniform solids layer deposition methods include preparing micro quantities of specimens for analysis of nucleotide probes by the polymerase chain reaction (PCR) method.

In embodiments, a method of reducing thickness non-uniformity in a microjet-deposited solids layer, comprising depositing onto a substrate by microjet deposition a composition comprising a liquid vehicle, at least one reagent; and a polyol, to form a uniformly thick solids layer comprising said at least one reagent on said substrate. The polyol is present at a concentration that enhances thickness uniformity of a solids layer containing said reagent, when said solids layer is formed from a microjet-deposited quantity of said composition on a substrate. For example, the polyol is preferably present in the composition at a concentration in the range of about 0.1% to about 5% (by weight), more preferably in the range of about 0.1% to about 3%. In embodiments, the method also includes drying the microjet-deposited composition, to form a uniformly thick solids layer comprising said at least one reagent on said substrate. Preferably the polyol is selected from the group consisting of polyols containing 3-12 carbon atoms substituted with 3-9 hydroxyl groups, such as sorbitol, xylitol, mannitol, maltitol, xylose, glycerol, saccharose and trehalose, for example. In embodiments, the polyol concentration is such that it prevents cracking, lifting, bubbling, roughening, and peeling of the uniform solids layer formed from said composition by microjet deposition onto a substrate. In embodiments, the total solids loading of the ink composition is up to 20% by weight. In some embodiments, the total solids loading of the ink composition is less than 10% by weight. In some embodiments the total solids loading is less than 5% by weight.

In embodiments a method of preparing a test strip includes forming on a substrate, in the manner described above, a uniformly thick solids layer comprising at least one reagent, wherein at least one of the reagents is a reagent for a selected test (e.g., a glucose concentration test). In embodiments, the substrate is divided into a plurality of test strips, with each strip containing a portion of the uniformly thick solids layer. In embodiments, the test strip is prepared by additionally depositing by microjetting onto a different site on the substrate an additional composition comprising a chemical reagent, and then drying the deposited compositions.

In embodiments a test strip comprises a substrate including a flow path for a liquid test specimen (e.g., blood); and a reaction zone containing a uniformly thick solids layer that includes at least one chemical reagent and a polyol. The uniformly thick solids layer is prepared as described above. At least one of the chemical reagents is at least partially soluble in the liquid specimen and interacts with the test specimen, or a component in the test specimen. For example, a component of the test specimen chemically reacts with or binds a chemical reagent. In embodiments the uniformly thick solids layer is 0.5-10 micrometers thick. In embodiments the uniformly thick solids layer resists cracking and peeling. In embodiments the polyol is present in the microjetted composition at a concentration in the range of about 0.1% to about 5% (by weight). In embodiments the polyol is present in the microjetted composition at a concentration in the range of about 0.1% to about 3% (by weight). In embodiments, the test is a glucose test, and the reaction zone also comprises working electrode, a counter electrode and a dielectric or insulator. In embodiments, the microjetted composition comprises at least one enzyme for reacting with glucose in the liquid test specimen, an electron mediator, a buffer, a polymer, a surfactant, and a polyol. In embodiments, the test strip is configured similarly to a conventional glucose test strip except for a uniformly-thick solid reagent layer deposited as described herein.

In embodiments a microjettable glucose test reagent composition comprises a liquid vehicle (e.g., water) in which are dissolved one or more enzymes that catalyze the oxidation of glucose to gluconolactone; potassium ferricyanide and/or other electron transfer agent; one or more buffers; one or more polymers; one or more surfactants; and 0.1-5% (by weight) of one or more polyols selected from the group of polyols containing 3-12 carbon atoms substituted with 3-9 hydroxyl groups. In embodiments, the polyol is present in the amount of about 0.1-3% by weight. In embodiments, the total solids loading in the glucose test reagent is up to 20% by weight. In embodiments the total solids loading of the of less than 10% by weight. In some embodiments the total solids loading is less than 5%.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications, including all equivalents of the subject matter of the claims.

What is claimed is:

1. A method of reducing thickness non-uniformity in a microjet-deposited solids layer, comprising:
depositing onto a substrate by microjet deposition a composition comprising
a liquid vehicle,
at least one reagent; and
a polyol, wherein said polyol is present at a concentration that enhances thickness uniformity of a solids layer containing said reagent, when said solids layer is formed from a microjet-deposited quantity of said composition on a substrate,
to form a uniformly thick solids layer comprising said at least one reagent on said substrate.

2. The method of claim 1 further comprising drying the microjet deposited composition to form a uniformly thick solids layer comprising said at least one reagent on said substrate.

3. The method of claim 1 wherein said polyol is present in the composition at a concentration in the range of about 0.1% to about 5% (by weight).

4. The method of claim 1 wherein said polyol is present in the composition at a concentration in the range of about 0.1% to about 3% (by weight).

5. The method of claim 1 wherein the polyol is selected from the group consisting of polyols containing 3-12 carbon atoms substituted with 3-9 hydroxyl groups.

6. The method of claim 1 wherein the polyol is selected from the group consisting of sorbitol, xylitol, mannitol, maltitol, xylose, glycerol, saccharose and trehalose.

7. The method of claim 1, wherein said polyol concentration is such that said solids layer resists cracking, lifting, bubbling, roughening, and peeling.

8. The method of claim 1 wherein the composition contains a total solids loading of no more than 20% by weight.

9. The method of claim 1 wherein the composition contains a total solids loading of less than 10% by weight.

10. The method of claim 1 wherein the composition contains a total solids loading of less than 5% by weight.

11. A method of preparing a test strip, comprising:
forming on a substrate, in accordance with the method of claim 1, a uniformly thick solids layer comprising at least one reagent, wherein at least one said reagent comprises a reagent for a selected test.

12. The method of claim 10, wherein said composition is a first composition, and the method further comprises:
depositing by microjetting onto said substrate an additional composition comprising a chemical reagent, wherein said additional composition is deposited at a different site on said substrate than said first composition; and
drying said microjetted compositions to form respective uniformly thick solids layers comprising the respective chemical reagents.

13. A test strip comprising:
a substrate strip comprising a flow path for a liquid test specimen;
a reaction zone comprising a uniformly thick solids layer containing at least one chemical reagent and a polyol, wherein said solids layer is prepared in accordance with the method of claim 1,
wherein said solids layer is disposed in said reaction zone, and
wherein at least one said reagent is at least partially soluble in said liquid and interacts with a test specimen, or a component thereof, when combined with said test specimen.

14. The test strip of claim 13 wherein said reaction zone is configured for allowing the interaction of at least one said reagent with said test specimen.

15. The test strip of claim 13 wherein said uniformly thick solids layer is 0.5-10 micrometers thick.

16. The test strip of claim 13 wherein said uniformly thick solids layer resists cracking and peeling.

17. The test strip of claim 13 wherein said polyol is present at a concentration in the range of about 0.1% to about 5% (by weight).

18. The test strip of claim 13 wherein said polyol is present at a concentration in the range of about 0.1% to about 3% (by weight).

19. The test strip of claim 13 wherein said test is a glucose test, said reaction zone further comprises a working electrode, a counter electrode and a dielectric, and said strip further comprises glucose meter contacts and electronic circuitry configured for coupling to said reaction zone and to said glucose meter contacts.

20. The test strip of claim 19 wherein said uniformly thick solids layer comprises at least one electron mediator, at least one buffering agent, at least one polymer, at least one surfactant, at least one polyol, and at least one enzyme that reacts with glucose in said liquid test specimen.

21. A microjettable glucose test reagent composition comprising:
a liquid vehicle;
an enzyme capable of catalyzing the oxidation of glucose to gluconolactone;
an electron mediator;
a buffer;
a polymer;
a surfactant; and
about 0.1-about 5% (by weight) polyol selected from the group of polyols containing 3-12 carbon atoms substituted with 3-9 hydroxyl groups.

22. The composition of claim 21 wherein said polyol is in the range of about 0.1-about 3% (by weight).

23. The composition of claim 21 containing a total solids loading of up to 20% by weight.

24. The composition of claim 21 containing a total solids loading of less than 10% by weight.

25. The composition of claim 21 containing a total solids loading of less than 5% by weight.

26. The composition of claim 21 wherein said liquid vehicle comprises water.
